(19) United States
(12) Patent Application Publication
(10) Pub. No.: US 2017/0095641 A1
(43) Pub. Date: Apr. 6, 2017

(54) CATHETER WITH CURVILINEAR POLYGON CROSS-SECTIONAL SHAPE

(71) Applicant: Covidien LP, Mansfield, MA (US)

(72) Inventors: Frank Scarpine, Brea, CA (US); Maria De Jesus Sanson, San Clemente, CA (US); Francis Bernard, Dana Point, CA (US); Rick Williams, Laguna Niguel, CA (US)

(21) Appl. No.: 14/875,740

(22) Filed: Oct. 6, 2015

(51) Int. Cl. A61M 25/00; A61M 25/01; A61M 25/09

(52) U.S. Cl. CPC A61M 25/0032; A61M 25/0043; A61M 25/0009; A61M 25/09; A61M 25/0097; A61M 25/0108; A61M 2025/0059

(57) ABSTRACT

In some examples, a catheter may include a catheter body configured such that at least one of a proximal portion or a medial portion of the catheter body has a cross-sectional shape including at least three apexes, adjacent apexes being connected by curvilinear sidewalls, the cross-section being taken substantially orthogonal to a longitudinal axis of the catheter body. In some examples, the entire catheter body has the cross-sectional shape including at least three apexes, adjacent apexes being connected by curvilinear sidewalls. In other examples, only a portion of the catheter body has the cross-sectional shape.

### TECHNICAL FIELD

This disclosure relates to a medical catheter.

### BACKGROUND

A medical catheter defining at least one lumen has been proposed for use with various medical procedures. For example, in some cases, a medical catheter may be used to access and treat defects in blood vessels, such as, but not limited to, lesions or occlusions in blood vessels.

### SUMMARY

In some aspects, this disclosure describes examples catheters that each include a catheter body configured such that at least one of a proximal portion or a medial portion of the catheter body has a cross-sectional shape including at least three apexes, adjacent apexes being connected by curvilinear sidewalls, the cross-section being taken substantially orthogonal to a longitudinal axis of the catheter body. In some examples, the entire catheter body has the cross-sectional shape including at least three apexes, adjacent apexes being connected by curvilinear sidewalls. In other examples, only a portion of the catheter body has the cross-sectional shape. Also described herein are methods of forming a catheter include a catheter body configured such that at least one of a proximal portion or a medial portion of the catheter body has the cross-sectional shape, and a method of using the catheter.

Clause 1: In some examples, a catheter comprises a catheter body extending between a proximal end and a distal end and defining at least one inner lumen, the catheter body comprising a proximal portion including the proximal end, a distal portion including the distal end, and a medial portion between the proximal and distal portions, wherein a cross-section of at least one of the proximal portion or the medial potion, taken substantially orthogonal to a longitudinal axis of the catheter body, has a shape including at least three apexes, adjacent apexes being connected by curvilinear sidewalls.

Clause 2: In some examples of the catheter of clause 1, the at least three apexes is three apexes.

Clause 3: In some examples of the catheter of clause 1 or 2, the at least three apexes is four, five, six, seven or eight apexes.

Clause 4: In some examples of the catheter of any of clauses 1-3, the shape is substantially symmetrical about the longitudinal axis of the catheter body.

Clause 5: In some examples of the catheter of any of clauses 1-4, the curvilinear sidewalls are shaped substantially as arcs.

Clause 6: In some examples of the catheter of any of clauses 1-5, each curvilinear sidewall is located between two of the apexes, and bows radially outwardly from the two apexes.

Clause 7: In some examples of the catheter of any of clauses 1-6, an entire length of the catheter body has a cross-sectional shape including at least three apexes, adjacent apexes being connected by curvilinear sidewalls.

Clause 8: In some examples of the catheter of any of clauses 1-7, a cross-sectional shape of the distal portion of the catheter body, taken substantially orthogonal to the longitudinal axis of the catheter body, has the shape including the at least three apexes, adjacent apexes being connected by curvilinear sidewalls

Clause 9: In some examples of the catheter of any of clauses 1-8, a cross-section of the at least one lumen has the shape including at least three apexes, adjacent apexes being connected by curvilinear sidewalls.

Clause 10: In some examples of the catheter of any of clauses 1-9, the catheter comprises a first catheter comprising a first catheter body, the first catheter body defining a protrusion extending into an inner lumen of the at least one inner lumen, the first catheter body being configured to receive second and third catheter bodies within the inner lumen on opposite sides of the protrusion.

Clause 11: In some examples of the catheter of any of clauses 1-10, the catheter body comprises an inner liner, a structural support member, and an outer jacket, wherein the structural support member is positioned between the inner liner and the outer jacket.

Clause 12: In some examples of the catheter of any of clauses 1-11, the proximal, distal, and medial portions have substantially equal lengths.

Clause 13: In some examples, a system comprises a first catheter comprising a first catheter body extending between a proximal end and a distal end and defining a first inner lumen, the first catheter body comprising a proximal portion including the proximal end, a distal portion including the distal end, and a medial portion between the proximal and distal portions, wherein a cross-section of at least one of the proximal portion or the medial potion, taken substantially orthogonal to a longitudinal axis of the catheter body, has a shape including at least three apexes, adjacent apexes being connected by curvilinear sidewalls; and a second catheter comprising a second catheter body defining a second inner lumen configured to receive the first catheter body.

Clause 14: In some examples of the system of clause 13, the proximal, distal, and medial portions of the first catheter are first proximal, medial, and distal portions, respectively, the proximal and distal ends of the first catheter body are first proximal and distal ends, respectively, and the second catheter body comprises a second proximal portion including a second proximal end of the second catheter body, a second distal portion including a second distal end of the second catheter body, and a second medial portion between the second proximal and distal portions, wherein a cross-section of at least one of the second proximal portion or the second medial portion, taken substantially orthogonal to a longitudinal axis of the second catheter body, has a substantially circular shape.

Clause 15: In some examples of the system of clause 13, the proximal, distal, and medial portions of the first catheter are first proximal, medial, and distal portions, respectively, the proximal and distal ends of the first catheter body are first proximal and distal ends, respectively, and the second catheter body comprises a second proximal portion including a second proximal end of the second catheter body, a second distal portion including a second distal end of the second catheter body, and a second medial portion between the second proximal and distal portions, wherein a cross-section of at least one of the second proximal portion or the second medial portion, taken substantially orthogonal to a longitudinal axis of the second catheter body, has the shape including at least three apexes, adjacent apexes being connected by curvilinear sidewalls.

Clause 16: In some examples of the system of any of clauses 13-15, the at least three apexes is three apexes.

Clause 17: In some examples of the system of any of clauses 13-16, the at least three apexes is four, five, six, seven, or eight apexes.

Clause 18: In some examples of the system of any of clauses 13-17, the shape is substantially symmetrical about the longitudinal axis of the first catheter body.

Clause 19: In some examples of the system of any of clauses 13-18, an entire length of the first catheter body has a cross-sectional shape including at least three apexes, adjacent apexes being connected by curvilinear sidewalls.

Clause 20: In some examples of the system of any of clauses 13-19, a cross-sectional shape of the distal portion of the first catheter body, taken substantially orthogonal to the longitudinal axis of the catheter body, has the shape including the at least three apexes, adjacent apexes being connected by curvilinear sidewalls.

Clause 21: In some examples of the system of any of clauses 13-20, a cross-section of the first inner lumen has the shape including at least three apexes, adjacent apexes being connected by curvilinear sidewalls.

Clause 22: In some examples, the system of any of clauses 13-21 further comprises a third catheter comprising a third catheter body, wherein the second catheter body defines a protrusion extending into the second inner lumen, the second catheter body being configured to receive the first and third catheter bodies within the second inner lumen on opposite sides of the protrusion.

Clause 23: In some examples of the system of any of clauses 13-22, the curvilinear sidewalls are shaped substantially as arcs.

Clause 24: In some examples of the system of any of clauses 13-23, each curvilinear sidewall is located between two of the apexes, and bows radially outwardly from the two apexes.

Clause 25: In some examples of the system of any of clauses 13-24, the first catheter body comprises: an inner liner; a structural support member; and an outer jacket, wherein the structural support member is positioned between the inner liner and the outer jacket.

Clause 26: In some examples, a method comprises positioning an inner liner over a mandrel, wherein a cross-section of at least one of a proximal portion of the mandrel or a medial portion of the mandrel, taken substantially orthogonal to a longitudinal axis of the mandrel, has a shape including at least three apexes, adjacent apexes being connected by curvilinear sidewalls, the mandrel comprising the proximal portion including a proximal end of the mandrel, a distal portion including a distal end of the mandrel, and the medial portion between the proximal and distal portions; positioning a structural support member over the inner liner; and positioning an outer jacket over the structural support member.

Clause 27: In some examples of the method of clause 26, the at least three apexes is three apexes.

Clause 28: In some examples of the method of clause 26 or clause 27, the at least three apexes is four, five, six, seven or eight apexes.

Clause 29: In some examples of the method of any of clauses 26-28, the shape is substantially symmetrical about the longitudinal axis of the mandrel.

Clause 30: In some examples of the method of any of clauses 26-29, an entire length of the mandrel has a cross-sectional shape including at least three apexes, adjacent apexes being connected by curvilinear sidewalls.

Clause 31: In some examples of the method of any of clauses 26-30, a cross-section of the distal portion of the mandrel, taken substantially orthogonal to the longitudinal axis of the mandrel, has a substantially circular shape.

Clause 32: In some examples of the method of any of clauses 26-31, a cross-sectional shape of the distal portion of the mandrel, taken substantially orthogonal to the longitudinal axis of the mandrel, has the shape including the at least three apexes, adjacent apexes being connected by curvilinear sidewalls.

Clause 33: In some examples of the method of any of clauses 26-32, the mandrel defines an indentation configured to define a protrusion along at least a portion of the inner lumen.

Clause 34: In some examples, a method comprises introducing a catheter body into a patient, the catheter body extending between a proximal end and a distal end and defining at least one inner lumen, the catheter body comprising a proximal portion including the proximal end, a distal portion including the distal end, and a medial portion between the proximal and distal portions, wherein a cross-section of at least one of the proximal portion or the medial potion, taken substantially orthogonal to a longitudinal axis of the catheter body, has a shape including at least three apexes, adjacent apexes being connected by curvilinear sidewalls; and guiding the distal end of the catheter body to a treatment site within the patient.

Clause 35: In some examples, the method of clause 34 further comprises introducing a guidewire in the patient, wherein guiding the catheter body to the treatment site comprises guiding the catheter body to the treatment site over the guidewire.

Clause 36: In some examples of the method of clause 34 or clause 35, the catheter comprises a first catheter including a first catheter body, and the method further comprises introducing a second catheter comprising a second catheter body in the patient, wherein guiding the first catheter body to the treatment site comprises guiding the first catheter body to the treatment site over the second catheter body.

Clause 37: In some examples of the method of any of clauses 34-36, the proximal, distal, and medial portions of the first catheter are first proximal, medial, and distal portions, respectively, the proximal and distal ends of the first catheter body are first proximal and distal ends, respectively, and the second catheter body comprises a second proximal portion including a second proximal end of the second catheter body, a distal portion including a second distal end of the second catheter body, and a second medial portion between the second proximal and distal portions, wherein a cross-section of at least one of the second proximal portion or the second medial portion, taken substantially orthogonal to a longitudinal axis of the second catheter body, has a substantially circular shape.

Clause 38: In some examples of the method of any of clauses 34-36, the proximal, distal, and medial portions of the first catheter are first proximal, medial, and distal portions, respectively, the proximal and distal ends of the first catheter body are first proximal and distal ends, respectively, and the second catheter body comprises a second proximal portion including a second proximal end of the second catheter body, a distal portion including a second distal end of the second catheter body, and a second medial portion between the second proximal and distal portions, wherein a cross-section of at least one of the second proximal portion or the second medial portion, taken substantially orthogonal to a longitudinal axis of the second catheter body, has the shape including at least three apexes, adjacent apexes being connected by curvilinear sidewalls.

Clause 39: In some examples of the method of any of clauses 34-38, the catheter comprises a first catheter including a first catheter body, and the method further comprises introducing a second catheter body into an inner lumen of the at least one inner lumen of the first catheter body.

Clause 40: In some examples of the method of clause 39, the proximal, distal, and medial portions of the first catheter are first proximal, medial, and distal portions, respectively, the proximal and distal ends of the first catheter body are first proximal and distal ends, respectively, and the second catheter body comprises a second proximal portion including a second proximal end of the second catheter body, a distal portion including a second distal end of the second catheter body, and a second medial portion between the second proximal and distal portions, wherein a cross-section of at least one of the second proximal portion or the second medial portion, taken substantially orthogonal to a longitudinal axis of the second catheter body, has the shape including at least three apexes, adjacent apexes being connected by curvilinear sidewalls.

Clause 41: In some examples of the method of clause 39, the proximal, distal, and medial portions of the first catheter are first proximal, medial, and distal portions, respectively, the proximal and distal ends of the first catheter body are first proximal and distal ends, respectively, and the second catheter body comprises a second proximal portion including a second proximal end of the second catheter body, a distal portion including a second distal end of the second catheter body, and a second medial portion between the second proximal and distal portions, wherein a cross-section of at least one of the second proximal portion or the second medial portion, taken substantially orthogonal to a longitudinal axis of the second catheter body, has a substantially circular shape.

Clause 42: In some examples, the method of clause 39, further comprises introducing a third catheter body into the inner lumen adjacent to the second catheter body, the second and third catheter bodies each having substantially circular cross-sectional shapes.

Clause 43: In some examples of the method of clause 42, the first catheter body defines a protrusion extending into the inner lumen, and introducing the second and third catheter bodies into the inner lumen comprises introducing the second and third catheter bodies into the inner lumen on opposite sides of the protrusion.

Clause 44: In some examples of the method of any of clauses 34-43, the at least three apexes is three apexes.

Clause 45: In some examples of the method of any of clauses 34-44, the at least three apexes is four, five, six, seven or eight apexes.

Clause 46: In some examples of the method of any of clauses 34-45, the shape is substantially symmetrical about the longitudinal axis of the catheter body.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a side elevation view of an example catheter, which includes a catheter body and a hub.

FIG. 2 is a perspective view of a distal portion of the catheter body of FIG. 1.

FIG. 3 is a conceptual cross-sectional view of the catheter body of FIG. 1 taken along line A-A in FIG. 1.

FIG. 4 is a perspective view of an example assembly including the catheter of FIG. 1 and another catheter.

FIG. 5 is a perspective view of another example assembly including the catheter of FIG. 1 and another catheter, the assembly being positioned at least partially within a vessel of a patient.

FIG. 6 is a conceptual cross-sectional view of the vessel and assembly of FIG. 5.

FIG. 7 is a conceptual cross-sectional view of an example assembly that includes two catheters each having a circular cross-sectional shape, the assembly being positioned at least partially within a vessel of a patient.

FIG. 8 is a conceptual cross-sectional view of another example catheter body.

FIG. 9 is a conceptual cross-sectional view of an example catheter body that includes a protrusion extending into an inner lumen.

FIG. 10 is a flow diagram of an example method of forming the catheter of FIG. 1.

### DETAILED DESCRIPTION

In some examples, a medical catheter (“catheter”) described herein includes a catheter body configured such that at least one of a proximal portion or a medial portion of the catheter body has a cross-sectional shape including at least three apexes, adjacent apexes being connected by curvilinear sidewalls, the cross-section being taken substantially orthogonal to a longitudinal axis of the catheter body. The shape including at least three apexes, adjacent apexes being connected by curvilinear sidewalls may also be referred to herein as, for example, a curvilinear polygon. In some examples, the entire catheter body has the curvilinear polygon cross-sectional shape. In other examples, only a portion of the catheter body has the curvilinear polygon cross-sectional shape (e.g., only the medial portion, only the proximal portion, only the medial and proximal portions, or only one of the medial or proximal portions in combination with the distal portion). In either example, however, the catheter body may be referred to as having a curvilinear polygon cross-sectional shape.

In some examples, proximal, medial, and distal portions of a catheter body have substantially equal lengths, the lengths being measured along a longitudinal axis of the catheter body. In other examples, at least two of the proximal, medial, and distal portions of a catheter body may have different lengths. The proximal portion may include a proximal end of the catheter body, the distal portion may include the distal end of the catheter body, and the medial portion may be positioned between the proximal and distal portions.

A catheter body with a curvilinear polygon cross-sectional shape may retain the strength and flexibility of a catheter body having a circular cross-sectional shape, but may allow for improved blood flow past the catheter body when the catheter body is introduced in vasculature of a patient. For example, for a given maximum cross-sectional dimension, a catheter body may with a curvilinear polygon cross-sectional shape may occupy less cross-sectional area within the vasculature than a catheter body having a circular cross-sectional shape, due at least in part to the configuration of the walls of the catheter body in the portion having the curvilinear polygon cross-sectional shape. Improving the blood flow past the catheter body within the vasculature may reduce the impact the catheter body has on the patient's circulation when the catheter is introduced within the patient's vasculature. The vasculature can comprise the neurovasculature, peripheral vasculature, or cardiovasculature.

In some cases, a clinician may rotate a catheter body in order to help steer the catheter body through vasculature of a patient. A distal portion of the catheter body leads a proximal portion of the catheter body through the vasculature, and may, therefore, be introduced in the patient while the proximal portion is external to the patient. The clinician may apply torque to at least one of a proximal portion or a medial portion of the catheter body in order to rotate the distal portion of the catheter. The catheter body described herein may exhibit improved torqueability relative to a catheter body having a circular cross-sectional shape along the entire length of the catheter body. For example, a catheter body including proximal and/or medial portions having a curvilinear polygon cross-sectional shapes may better transmit torques to the distal portion of the catheter body, and may be more resistant to kinking upon rotation of catheter body from the relatively proximal portion of the catheter body compared to the catheters that are circular in cross-section along the proximal and/or medial portions. Better torqueability may contribute to easier navigability of the catheter body, e.g., through tortuous vasculature in a brain of the patient.

The catheters described herein may be advanced to a target tissue site (or “target location”) within vasculature of the patient in cooperation with a guidewire, an inner catheter, or both, which may aid in the navigation (e.g., steering and manipulation) of the catheter through the vasculature. For example, an inner lumen of the catheter body may be configured to receive a guidewire or an inner catheter, such that the catheter body may be guided through vasculature over the guidewire or the inner catheter. In other examples, the catheters described herein may help guide another catheter to a target location within the vasculature, e.g., the catheters may be configured to be received in a lumen of another catheter.

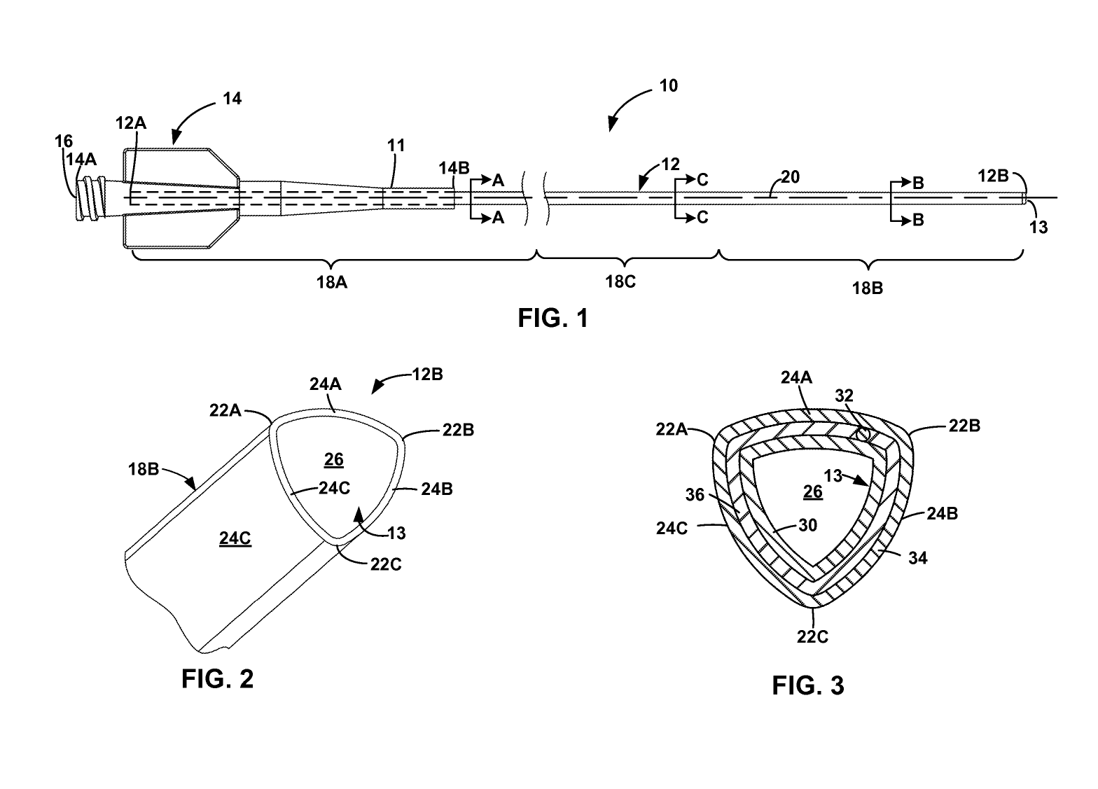

FIG. 1 is a side elevation view of an example catheter 10, which includes catheter body 12 and hub 14. Catheter hub 14 is positioned at a proximal end of catheter 10 and defines an opening 16 through which an inner lumen of catheter body 12 may be accessed and, in some examples, closed. For example, catheter hub 14 may include a luer connector for connecting to another device, a hemostasis valve, or another mechanism or combination of mechanisms. In some examples, catheter 10 includes strain relief member 11, which may be a part of hub 14 or may be separate from hub 14. In other examples, the proximal end of catheter 10 can include another structure in addition or, or instead of, hub 14.

Catheter body 12 is a flexible elongated body that extends from proximal end 12A to distal end 12B and defines at least one inner lumen (e.g., one inner lumen, two inner lumens, or three or more inner lumens) that terminates at distal opening 13 defined by catheter body 12. In some examples, the flexible catheter body 12 is configured to substantially conform to the curvature of the vasculature when introduced in the vasculature. In the example shown in FIG. 1, proximal end 12A of catheter body 12 is received within hub 14 and is mechanically connected to hub 14 via an adhesive, welding, or another suitable technique or combination of techniques. Opening 16 defined by hub 14 and located at proximal end 14A of hub 14 is aligned with the inner lumen of catheter body 12, such that the inner lumen of catheter body 12 may be accessed via opening 16.

Catheter body 12 defines proximal portion 18A that includes proximal end 12A of catheter body 12, distal portion 18B that includes distal end 12B of catheter body 12, and medial portion 18C, which is positioned between and directly adjacent to proximal portion 18A and distal portion 18B. Portions 18A-18C may have any suitable length relative to each other, the length being measured along longitudinal axis 20 of catheter body 12. In some examples, portions 18A-18C have substantially equal (e.g., equal or nearly equal) lengths. In other examples, one portion may have a greater length than another portion. For example, proximal portion 18A may have a greater length than the medial portion 18C and/or the distal portion 18B. As another example, medial portion 18C may have a greater length that proximal portion 18A and/or distal portion 18B.

The lengths of each of the portions 18A-18C, and, therefore, the length of catheter body 12, may be selected to be suitable for accessing a target location within the patient from a vascular access point. The target location may depend on the medical procedure for which catheter 10 is used. For example, if catheter 10 is a distal access catheter used to access vasculature in a brain of a patient from a femoral artery access point at the groin of the patient, catheter body 12 may have a length of about 129 centimeters (cm) to about 135 cm, such as about 132 cm, although other lengths may be used.

Although primarily described as being used to reach relatively distal vasculature sites, the catheters with curvilinear polygon cross-sectional shapes described herein, including catheter 10, may readily be configured to be used with other target tissue sites. For example, the catheters may be used to access tissue sites throughout the coronary and peripheral vasculature, the gastrointestinal tract, the urethra, ureters, Fallopian tubes and other body lumens.

At least one of proximal portion 18A or medial portion 18C of catheter body 12 has a cross-sectional shape including at least three apexes, adjacent apexes being connected by curvilinear sidewalls, i.e., a curvilinear polygon shape. The cross-section is taken substantially orthogonal to longitudinal axis 20. In some examples, such as the one shown in FIGS. 1-6, proximal, distal, and medial portions 18A-18C each have the curvilinear polygon cross-sectional shape, such that an entire length of catheter body 12 has the curvilinear polygon cross-sectional shape. In some examples, the cross-sections of proximal, distal, and medial portions 18A-18C have substantially the same dimensions. In other examples, the cross-sections of two or more of the proximal, distal, and medial portions 18A-18C have different dimensions. For example, proximal portion 18A may have a greater maximum cross-sectional dimension (e.g., measured between apexes) than distal portion 18B.

A larger maximum cross-sectional dimension at proximal portion 18A may provide better proximal support for catheter body 12, which may help increase the pushability of catheter body 12. In addition, a smaller maximum dimension at distal portion 18B may help increase the flexibility of catheter body 12 along distal portion 18B. A catheter body having a smaller maximum dimension (e.g., an outer diameter in the case of a catheter body having a circular cross-section) may be easier to navigate through tortuous vasculature. Thus, by reducing the maximum cross-sectional dimension of catheter body 12 at distal portion 18B, which leads catheter body 12 through vasculature, catheter body 12 may better traverse through tortuous vasculature with still maintaining a relatively high level of proximal pushability. In some cases, proximal portion 18A may not be introduced into low profile or tortuous arteries, such that the cross-sectional size of proximal portion 18A may be increased in favor of proximal support without adversely affecting the ability of catheter body 12 to reach relatively distal tissue sites.

In some examples in which proximal portion 18A has a larger maximum cross-sectional dimension than distal portion 18B, medial portion 18C may gradually taper from a first maximum cross-sectional dimension of proximal portion 18A to a second maximum cross-sectional dimension of distal portion 18B. Thus, medial portion 18C can define a smooth transition from proximal portion 18A to distal portion 18B. In some examples, medial portion 18C continuously tapers (e.g., a linear rate of change in outer diameter) from the first maximum cross-sectional dimension to the second maximum cross-sectional dimension. In other examples, medial portion 18C may taper in a curved manner, e.g., defining a convex or concave curve, or it may progressively change in outer diameter, e.g., it may define discrete step-downs in cross-sectional dimension to define the taper. The size of the discrete step-downs in cross-sectional dimension may be selected to reduce the number of edges that may catch on anatomical features within the vasculature as catheter body 12 is advanced through vasculature.

In other examples of catheter 10, only one of proximal portion 18A or medial portion 18C may have the curvilinear polygon cross-sectional shape, and the other of proximal portion 18A or medial portion 18C may have a substantially circular (e.g. circular or nearly circular) cross-sectional shape. In these examples, distal portion 18B may also have the curvilinear polygon cross-sectional shape or may have a substantially circular cross-sectional shape. In some applications of catheter 10, distal portion 18B having a substantially circular cross-sectional shape may provide advantages. For example, such a distal portion 18B may define a part of lumen 26 having a substantially circular cross-sectional shape, which, for a given maximum cross-sectional dimension, may be larger in cross-sectional area than a part of inner lumen 26 having a curvilinear polygon cross-sectional shape. A relatively large inner lumen at distal end 12B of catheter body 12 may provide for more efficient and/or more effective aspiration of thrombus from vasculature compared to catheter bodies having smaller inner lumens at the distal end, e.g., due to a larger aspiration force that can be applied to catheter 10, due to the larger inner lumen portion for receiving the thrombus, or both.

FIG. 2 is a perspective view of distal end 12B of catheter body 12 and illustrates distal portion 18B of catheter body 12, which defines opening 13 to inner lumen 26 of catheter body 12. FIG. 3 is cross-sectional view of a portion of catheter body 12, which can be, for example, proximal portion 18A of catheter body 12 taken along lines A-A in FIG. 1, distal portion 18B of catheter body 12 taken along lines B-B in FIG. 1, and/or medial portion 18C of catheter body 12 taken along lines C-C in FIG. 1.

FIGS. 2 and 3 illustrate an example curvilinear polygon cross-sectional shape of catheter body 12. The example curvilinear polygon includes three apexes 22A-22C, adjacent apexes being connected by curvilinear sidewalls 24A-24C. In the example shown in FIG. 2, apexes 22A-22C are distributed substantially evenly (e.g., evenly or nearly evenly) radially around the outer perimeter of catheter body 12. As a result, curvilinear sidewalls 24A-24C have substantially identical (e.g., identical or nearly identical) lengths, the length of a sidewall 24A-24C being measured along the respective curvilinear sidewall between radially adjacent apexes. Because sidewalls 24A-24C are curvilinear, the lengths of the sidewalls may be referred to as arc lengths. In addition, due to the substantially even distribution of apexes 22A-22C around the outer perimeter of catheter body 12, the example cross-sectional shape shown in FIGS. 2 and 3 is substantially symmetric (e.g., symmetric or nearly symmetric), e.g., relative to a line that is perpendicular to longitudinal axis 20 (FIG. 1) and extends through a center of lumen 26 and through an apex.

Curvilinear sidewalls 24A-24C can each bow radially outward from a geometric center of catheter body 12 (in cross-section), which may be, for example, a center of inner lumen 26. Due at least in part to the bowed-out or radially outward extension of curvilinear sidewalls 24A-24C from the geometric center of catheter body 12, catheter body 12 with the depicted example curvilinear polygon cross-sectional shape may exhibit an increased inner cross-sectional dimension relative to a catheter body having a triangular cross-sectional having similarly spaced apexes joined by straight walls. A relatively large inner cross-sectional dimension may provide for a catheter body 12 defining a relatively large inner lumen 26 (also referred to as a working channel in some examples), through which distal tissue sites may be accessed, e.g., to deliver a medical device or therapeutic agent, to remove a thrombus or other target from the patient's body, or any combination thereof.

In other examples, the curvilinear polygon cross-sectional shape of catheter body 12 may include more than three apexes, adjacent apexes being connected by curvilinear walls. For example, catheter body 12 may include four, five, six, seven, eight or more apexes, adjacent apexes being connected by curvilinear walls. In contrast to a catheter body having a cross-sectional shape that includes any such number of apexes joined by straight walls, due at least in part to the radially outward extension of the curvilinear sidewalls of catheter body 12 from the geometric center of catheter body 12, catheter body 12 may have a larger inner lumen 26 for a given maximum distance between apexes (e.g., as measure by a straight line connecting the apexes).

In addition, the curved walls of catheter body 12 may define a relatively more atraumatic interface with adjacent tissue compared to a catheter body having a cross-sectional shape with three or more apexes connected by straight walls. As a result, catheter body 12 may exhibit fewer adverse interactions with vessel walls as the catheter body is navigated through curves in the patient's vasculature compared to the catheter body having apexes joined by straight walls.

As discussed above, in some examples, apexes 22A-22C may be substantially evenly distributed radially about an outer perimeter of catheter body 12. In other examples, apexes 22A-22C may have another arrangement relative to each other. For example, apexes 22A, 22B may be closer to each other than apexes 22A, 22C and apexes 22B, 22C, such that walls 24B, 24C have a greater length than wall 24A. The distances between apexes 22A-22C may be selected, e.g., to accommodate different target tissue sites within a patient.

Catheter body 12 is structurally configured to be relatively flexible, pushable, and relatively kink- and buckle-resistant, so that it may resist buckling when a pushing force is applied to a relatively proximal portion of the catheter to advance the catheter body distally through vasculature, and so that it may resist kinking when traversing around a tight turn in the vasculature. Kinking and/or buckling of catheter body 12 may hinder a clinician's efforts to push catheter body 12 distally, e.g., past a turn in a vessel. The curvilinear polygon cross-sectional shape of catheter body 12 may contribute to improved torqueability relative to a catheter body having a circular cross-sectional shape along the entire length of the catheter body. The improved torqueability may allow catheter body 12 to better resist kinking when a torsional force is applied to proximal portion 18A.

As shown in FIG. 3, in some examples, catheter body 12 includes inner liner 30 that defines inner lumen 26, outer jacket 34, and/or structural support member 32 positioned between inner liner 30 and outer jacket 34. Inner liner 30, structural support member 32, and outer jacket 34 interact to define a relatively flexible catheter body 12 with sufficient structural integrity (e.g., columnar strength) to permit catheter body 12 to be advanced through a patient's vasculature from a pushing force applied to proximal portion 18A of catheter body 12, without buckling or undesirable bending (e.g., kinking) of catheter body 12.

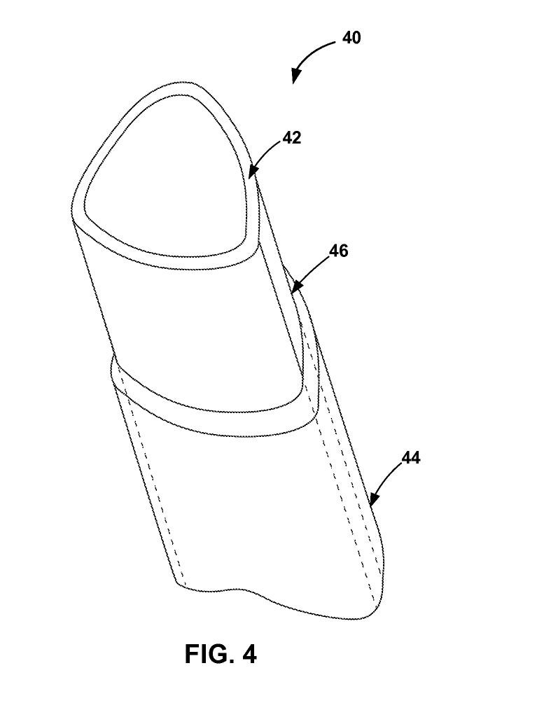

Inner liner 30 defines inner lumen 26 of catheter body 12, inner lumen 26 extending from proximal end 12A to distal end 12B and defining a passageway extending from proximal end 12A to distal opening 13 at distal end 12B of catheter body 12. As shown in FIGS. 2 and 3, in some examples, inner lumen 26 of catheter body 12 may have curvilinear polygon cross-sectional shape that corresponds to the outer curvilinear polygon cross-sectional shape of catheter body 12. In other examples, inner lumen 26 of catheter body 12 may also have a substantially circular (e.g., circular or nearly circular) cross-sectional shape. As discussed with respect to FIG. 4, in some examples, the outer perimeter of catheter body 12 that defines the curvilinear polygon cross-sectional shape may provide advantages, such that even if inner lumen 26 defines a substantially circular cross-sectional shape, catheter 10 may exhibit advantages over a catheter having an outer perimeter defining a substantially circular cross-sectional shape.

Inner lumen 26 may be sized to receive a medical device (e.g., another catheter, a guidewire, an embolic protection device, a stent, a thrombectomy device, a delivery system used in combination with any of the foregoing, or any combination thereof), a therapeutic agent, or the like. At least the inner surface of inner liner 30 defining inner lumen 26 may be lubricious in some examples in order to facilitate the introduction and passage of a device, a therapeutic agent, delivery system or the like, through inner lumen 26. For example, the material from which the entire inner liner 30 is formed may be lubricious, or inner liner 30 may be formed from two or more materials, where the material that defines inner lumen 26 may be more lubricious than the material that interfaces with structural support member 32. In addition to, or instead of, being formed from a lubricious material, in some examples, an inner surface of inner liner 30 is coated with a lubricious coating.

Example materials from which inner liner 30 may be formed include, but are not limited to, polytetrafluoroethylene (PTFE), fluoropolymer, perfluoroalkyoxy alkane (PFA), fluorinated ethylene propylene (FEP), or any combination thereof. For example, inner liner 30 may be formed from a non-etched PTFE, e.g., may consist essentially of a non-etched PTFE.

Structural support member 32 can extend along a length of catheter body 12, e.g., along the entire catheter body or only along a part of catheter body 12. For example, member 32 may extend along only a part of proximal portion 18A, only a proximal part of distal portion 18B, and along the entire medial portion 18C. Other configurations of structural support member 32 can also be used.

Structural support member 32 can be configured to increase the structural integrity of catheter body 12 while allowing catheter body 12 to remain relatively flexible. For example, member 32 may be configured to help catheter body 12 substantially maintain its cross-sectional shape or at least help prevent catheter body 12 from buckling or kinking as it is navigated through tortuous anatomy. Structural support member 32, together with inner liner 30 and outer jacket 34, may help distribute both pushing and rotational forces along a length of catheter body 12, which may help prevent kinking of body 12 upon rotation of body 12 or help prevent buckling of body 12 upon application of a pushing force to body 12. As a result, a clinician may apply pushing forces, rotational forces, or both, to a proximal portion of catheter body 12, and such forces may cause a distal portion of catheter body 12 to advance distally, rotate, or both, respectively.

In the example shown in FIG. 3, structural support member 32 includes a coil member defining a plurality of turns, e.g., in the shape of a helix. In other examples, structural support member 32 includes a generally tubular braided structure, or a combination of a braided structure and a coil member.

Outer jacket 34 can be positioned radially outward of inner liner 18 and structural support member 32, and, in some examples, defines an outer surface of catheter body 12. Although a coating or another material may be applied over the outer surface of outer jacket 34, outer jacket 34 may still substantially define shape and size of the outer surface of catheter body 12. Outer jacket 34, together with structural support member 32 and inner liner 30, may be configured to define catheter body 12 having the desired flexibility, kink resistance, and pushability characteristics.

Outer jacket 34 may have stiffness characteristics that contribute to the desired stiffness profile of catheter body 12. For example, outer jacket 34 may be formed to have a stiffness that decreases from proximal portion 18A of catheter body 12 to distal portion 18B. Outer jacket 34 can be formed of substantially the same material, or may be formed from two or more different materials that enable outer jacket 34 to exhibit the desired stiffness characteristics.

Example materials from which outer jacket 34 may be defined include, but are not limited to, polymers, such as a polyether block amide (e.g., PEBAX®, commercially available from Arkema Group of Colombes, France), an aliphatic polyamide (e.g., Grilamid®, commercially available from EMS-Chemie of Sumter, S.C.), another thermoplastic elastomer or other thermoplastic material, or combinations thereof.

In some examples, at least a portion of an outer surface of catheter body 12 (e.g., an outer surface of outer jacket 34) includes one or more coatings, such as, but not limited to, an anti-thrombogenic coating, which may help reduce the formation of thrombi in vivo, an anti-microbial coating, and/or a lubricating coating. The lubricating coating may be configured to reduce static friction and/ kinetic friction between catheter body 12 and tissue of the patient as catheter body 12 is advanced through the vasculature. The lubricating coating can be, for example, a hydrophilic coating. In some examples, the entire working length of catheter body 12 (from distal portion 14B of hub 14 to distal end 12B) is coated with the hydrophilic coating. In other examples, only a portion of the working length of catheter body 12 coated with the hydrophilic coating. This may provide a length of catheter body 12 distal to distal end 14B of hub 14 with which the clinician may grip catheter body 12, e.g., to rotate catheter body 12 or push catheter body 12 through vasculature.

In some examples, catheter body 12 includes support layer 36 between inner liner 30 and outer jacket 34. Structural support member 32 may couple, adhere, or otherwise mechanically connect to at least a portion of an outer surface of inner liner 30 via support layer 36. In some examples, support layer 36 is positioned between the entire length of structural support member 32 and inner liner 30. In other examples, support layer 36 is only positioned between a part of the length of structural support member 32 and inner liner 30.

Support layer 36 may be a thermoplastic material or a thermoset material, such as a thermoset polymer and/or a thermoset adhesive (e.g., a thermoset polyurethane adhesive, such as Flexobond 430, commercially available from Bacon Industries of Irvine, Calif.). In some cases, the material forming support layer 36 may have elastic properties, such that there may be a tendency for support layer 36 to a return to a resting position. This may be referred to as “bounce back” of the support layer. A support layer 36 formed from a cured thermoset polyurethane adhesive exhibits a relatively delayed bounce back response compared to a thermoplastic material, e.g., due at least in part to the elastic properties of the thermoset polyurethane adhesive. The delayed bounce back response may be advantageous for navigating catheter body 12 through vasculature. For example, the delayed bounce back response may reduce the extent to which catheter body 12 may spring against vascular walls as it is advanced through the vasculature.

In some examples, catheter 10, as well as other catheters described herein, may be a guide catheter that acts as a conduit to help support a microcatheter. In other examples, catheter 10 may be a microcatheter. In either example, catheter body 12 defines inner lumen 26, which may be configured to receive one or more medical devices and/or delivery systems used in combination therewith, deliver a therapeutic agent to a distal tissue site, remove thrombus (e.g., by aspiration) from the patient's vasculature, and the like or any combination thereof.

FIG. 4 is conceptual perspective view illustrating an example assembly 40 that includes first catheter 42 and second catheter 44, and illustrates first catheter 42 at least partially received in inner lumen 46 defined by second catheter 44. First catheter 42 and/or second catheter 44 may be examples of catheter 10 shown in FIGS. 1-3. First catheter 42 may be an inner catheter over which second catheter 44 may be guided to a target location within a patient, and/or second catheter 44 may define a conduit through which first catheter 42 is guided to a target location.

Second catheter 44 defines inner lumen 46 that is sized and configured to receive first catheter 42 Inner lumen 46 has a curvilinear polygon cross-sectional shape that is substantially similar (e.g., identical or nearly identical) to the curvilinear polygon cross-sectional shape of catheter body 12. Thus, when first catheter 42 is introduced inside of inner lumen 46, e.g., when a distal end of a catheter body of first catheter 42 is introduced into a proximal end of inner lumen 46, first and second catheters 42, 44 mate (or nest) together. The substantially similar cross-sectional shapes of first catheter 42 and inner lumen 46 of second catheter 44 may help to maintain the relative orientation of catheters 42, 44 when first catheter 42 and inner lumen 46 are sized relative to each other so that first catheter 42 may not rotate within inner lumen 46. The maintenance of the relative orientation of catheters 42, 44 may allow a clinician to maintain radial directional control of first catheter 42 when guiding catheter body 42 to a target location within the patient, which allows first catheter 42 (or second catheter 44) to be delivered to a target location within a patient with a particular region of the catheter facing the desired radial direction. This may be useful if, for example, the catheter being delivered has one or more side openings and it is desirable to control the radial orientation of the side openings within the patient.

As discussed above, in some examples, only a part of a catheter body, e.g., only one or both of a proximal portion or a medial portion, may have the curvilinear polygon cross-sectional shape. In these examples, the curvilinear polygon cross-sectional shape of the part of a first catheter body may still help maintain the relative orientation of the first catheter body with a second catheter body having a similar cross-sectional shape profile, the second catheter body being received within an inner lumen of the first catheter body or defining an inner lumen inside of which the first catheter body is received.

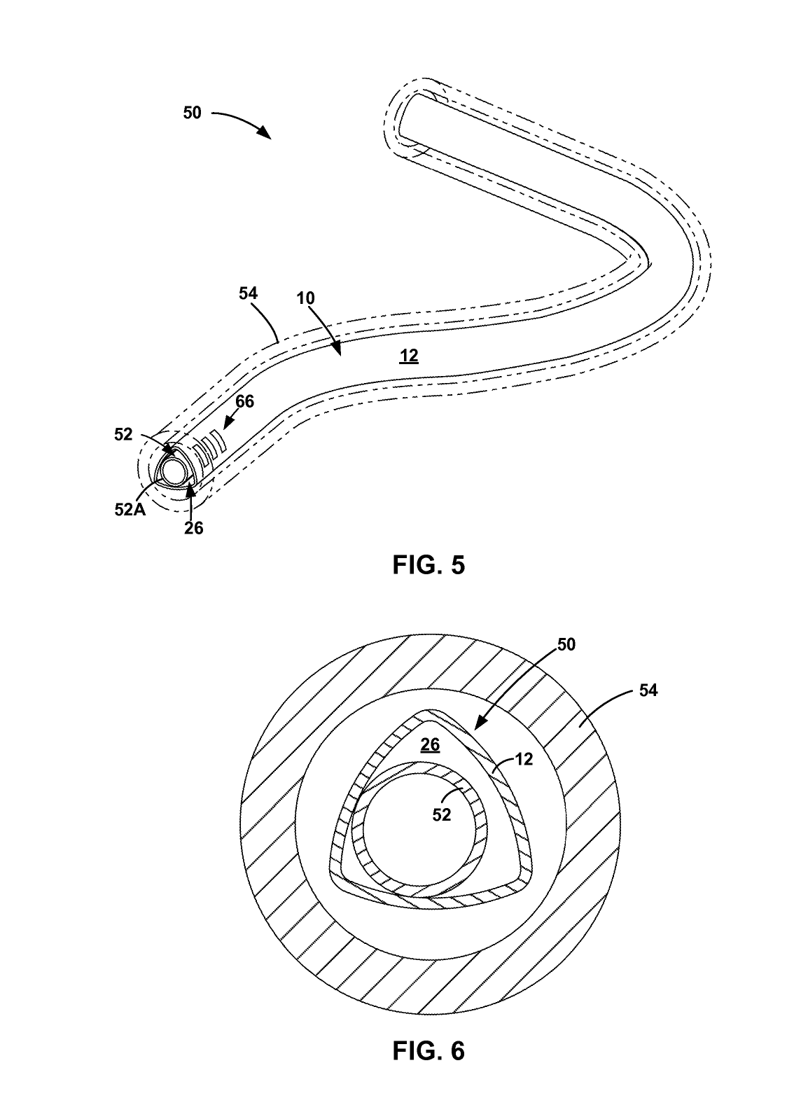

In some examples, a catheter with a curvilinear polygon cross-sectional shape and defining a lumen having a curvilinear polygon cross-sectional shape may be used to guide a catheter having a circular cross-sectional shape to a target tissue site within vasculature of a patient. FIG. 5 illustrates a conceptual perspective view of an example assembly 50 that includes catheter 52 having a circular cross-section introduced inside inner lumen 26 defined by catheter body 12 of catheter 10. FIG. 6 illustrates a conceptual cross-sectional view of assembly 50 and vasculature 54, taken substantially perpendicular (e.g., perpendicular or nearly perpendicular) to longitudinal axis 20 (FIG. 1) of catheter body 12 and through catheter 52 and vessel 54. In some examples, catheter body 12 may be disposed within vessel 54 of patient, as shown in FIGS. 5 and 6. The cross-sectional view of catheter body 12 does not illustrate inner liner 30, structural support member 32, outer jacket 34, and support layer 36.

In some examples, catheter body 12 may be navigated to target tissue site within vasculature 54 over catheter 52 that defines a substantially circular (e.g., circular or nearly circular) cross-sectional shape. In these examples, the friction between the catheter and inner catheter may be relatively low compared to examples in which the outer catheter is also round in cross-section along its entire length. Decreasing the friction between catheters 10, 52 may increase the speed with which catheter 10 is guided to the target tissue site over catheter 52, which may be desirable in the case of some medical procedures.

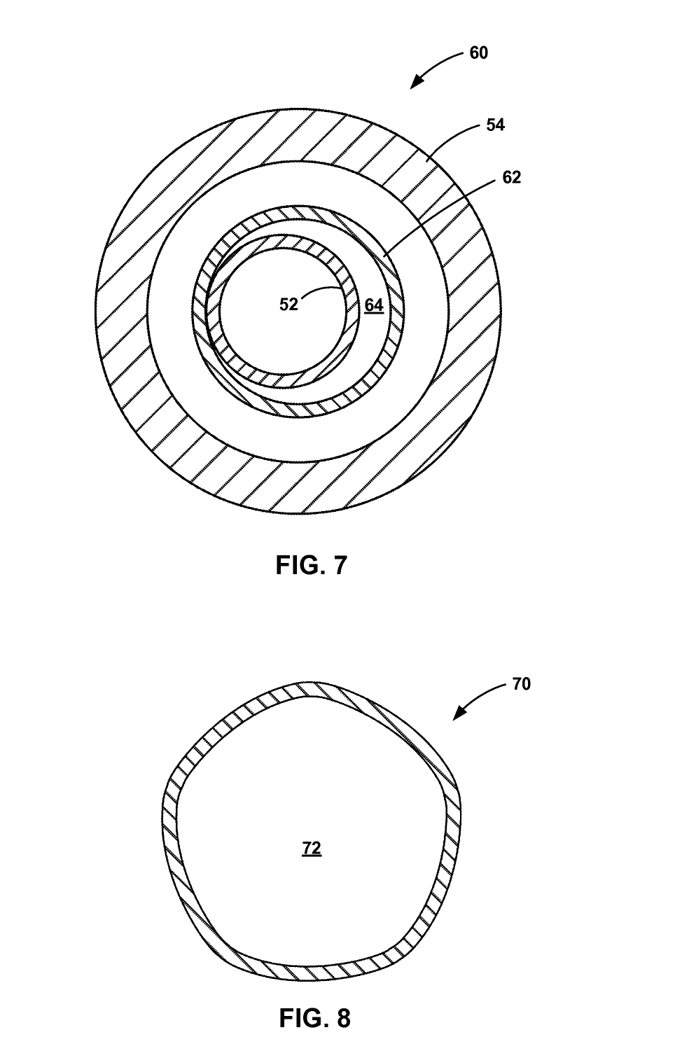

The lower friction between catheters 10, 52 may be at least partially attributable to the smaller surface area of inner lumen 26 of catheter 10 that is in contact with an outer surface of inner catheter 52 that results from inner lumen 26 having the curvilinear polygon cross-sectional shape. FIG. 7 is a cross-sectional view of an example assembly 60 including catheter 52 disposed within catheter 56 having a substantially circular cross-sectional shape, where the cross-section is taken substantially perpendicular to a longitudinal axis of catheter 52. As shown in FIG. 7, when catheter 52 is disposed within inner lumen 64 defined by catheter 62, inner lumen 64 having a substantially circular cross-sectional shape, a continuous curvilinear outer surface of inner catheter 52 engages with inner lumen 64 of catheter 56. In contrast, as shown in FIG. 6, when catheter 52 is disposed within inner lumen 26 having a curvilinear polygon shape, discontinuous portions of catheter 52 engage with catheter body 12 of catheter 10. Thus, for a given catheter 52 outer diameter and a given maximum diameter of inner lumen 26 and inner lumen 64, less surface area of catheter 52 may engage with catheter body 12 of catheter 10 than with catheter 62, thereby resulting in less sliding friction between catheter 52 and catheter 10, compared to catheter 52 and round catheter 62.

In some examples, catheter body 12 and/or catheter 52 may each include one or more radiopaque markers at or near the respective distal ends. A radiopaque marker can be, for example, a radiopaque marker band (e.g., a ring or one or more partial rings) attached to the respective catheter bodies, e.g., by an adhesive or held in place between an outer jacket and an inner liner. In addition to, or instead of a radiopaque marker band, as shown in FIG. 5, the radiopaque marker can include a plurality of grooves 66, which are shown in FIG. 5 to protrude from an outer surface of catheter body 12 or are defined by and recessed within an outer surface of catheter body 12. Grooves 66 may be, for example, a series of tangential arcs along an inner diameter of the catheter body. Grooves 66 may be formed from a radiopaque material, or may be filled with a radiopaque material in the case of recessed grooves, which may be visible within the patient with the aid of suitable medical imaging equipment.

Grooves 66 or another radiopaque marker may help a clinician determine an orientation of catheter body 12 within a patient or a relative orientation between catheters 10, 52. In addition to, or instead of being used to determine the orientation of catheter body 12, grooves 66 or another radiopaque marker may help the clinician align distal end 12B of catheter body 12 with distal end 52A of catheter 52.

FIG. 8 is a cross-sectional view of an example catheter body 70 that has a curvilinear polygon cross-sectional shape having more than three apexes, adjacent apexes being connected by curvilinear walls. Catheter body 70 may have other components, such as an inner liner, a structural support member, or an outer jacket, but these components are not shown in FIG. 8. The cross-sectional shape shown in FIG. 8 may be defined along an entire length of the catheter body, only along at least one of a proximal portion or a medial portion of catheter body 70, or along at least one of the proximal portion or the medial portion and a distal portion of catheter body 70. Catheter body 70 may have some of the same advantages of catheter body 12 described above, such as reducing friction with a catheter having a substantially circular cross-sectional shape and received in inner lumen 72, or being configured to maintain a relative orientation with an outer or inner catheter having a catheter body with a substantially similar curvilinear polygon cross-sectional shape.

In some examples, a catheter body with a curvilinear polygon cross-sectional shape can define an inner lumen (having the same or similar cross-sectional shape) that is configured to accommodate two or more catheters in an efficient manner. For example, compared to a catheter body defining an inner lumen having a circular cross-sectional shape and being sized to receive two or more catheters in a side-by-side configuration, a catheter body defining an inner lumen having a curvilinear polygon cross-sectional shape and also configured to receive the same two or more catheters in a side-by-side configuration may have a lower profile and occupy less room within a patient's blood vessel. In some examples, a catheter body defining an inner lumen having a curvilinear polygon cross-sectional shape may define at least one protrusion (e.g., one protrusion or two protrusions) extending into an inner lumen. The one or more protrusions may each function as an inner lumen divider to help keep two or more catheters separate from each other as they are guided through the inner lumen defined by the catheter body.

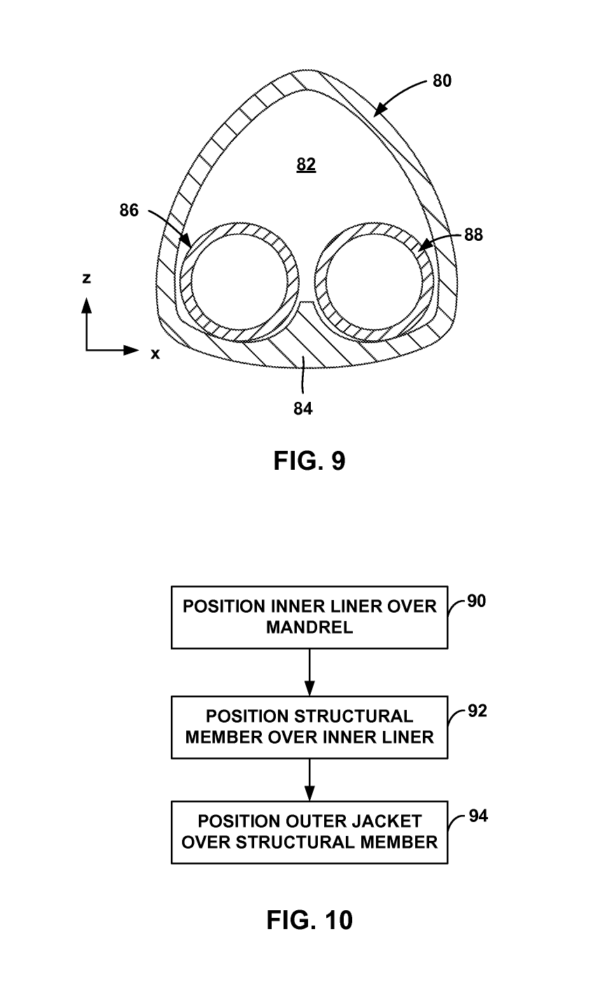

FIG. 9 is a conceptual cross-sectional view of an example catheter body 80, the cross-section being taken in the x-z plane (orthogonal x-z axes are shown in FIG. 9 to aid the description), which is substantially orthogonal to a longitudinal axis of catheter body 80. Catheter body 80 defines inner lumen 82 and includes protrusion 84 that extends into inner lumen 82. Protrusion 84 can be a separate component that is attached (e.g., via an adhesive or welding) inside inner lumen 82, or may be integrally formed with the rest of catheter body 80.

Catheter body 80 is configured to receive first inner catheter 86 within inner lumen 82 on a first side of protrusion 84 and a second inner catheter 88 within inner lumen 82 on a second, opposite side of protrusion 84. Protrusion 84 defines separate spaces for inner catheters 86, 88, and helps define a separate pathway for inner catheters 86, 88 through a common inner lumen which may help increase the speed with which a clinician guides first and second inner catheters 86, 88 through catheter body 80 to a target tissue site. For example, protrusion 84 may help prevent first and second inner catheters 86, 88 from crossing over each other as they are guided through inner lumen 82.

In some examples, protrusion 84 may extend along the entire length of inner lumen 82. In other examples, protrusion 84 may extend along only a part of the length of inner lumen 82. Protrusion 84 can be, for example, a continuous member that extends along the entire or partial length of catheter body 80, or protrusion 84 may be discontinuous along the length of catheter body 80.

Protrusion 84 can have any suitable height (measured in the z-axis direction), but does not extend across the entire width of inner lumen 84. In this way, protrusion 84 may differ from a septum that defines two lumens.

The catheters described herein can be formed using any suitable technique. FIG. 10 is a flow diagram of an example method of forming a catheter with a curvilinear polygon cross-sectional shape. Although FIG. 10 is described with respect to catheter body 12, in other examples, the technique shown in FIG. 10 may be used to form another catheter body having a curvilinear polygon cross-sectional shape.

In accordance with the technique shown in FIG. 10, inner liner 30 may be positioned over a mandrel (90), such as by inserting the mandrel through an end of inner liner 30. The mandrel may define an outer surface that has the desired cross-sectional shape of catheter body 12. For example, if the entire length of catheter body 12 (and inner lumen 26) has a curvilinear polygon cross-sectional shape, the mandrel may also define the curvilinear polygon cross-sectional shape. In contrast, if only a part of catheter body 12 has a curvilinear polygon cross-sectional shape, only a part of the mandrel may define the curvilinear polygon cross-sectional shape. The other part of the mandrel may define, for example, a circular cross-sectional shape.

The mandrel may be formed from any suitable material. The material from which the mandrel is formed may be configured to relatively easily release inner liner 30, e.g., after catheter body 12 is formed over the mandrel. For example, the mandrel may be formed from an extruded PTFE (e.g., the mandrel may consist of or consist essentially of an extruded PTFE). An extruded PTFE material may define a relatively lubricious outer surface, which may allow for relatively easy release of inner liner 30 from the mandrel, e.g., even in the absence of one or more additional lubricious coatings on the outer surface of the mandrel.

In some examples, in the technique shown in FIG. 10, after positioning inner liner 30 over the mandrel, inner liner 30 may be heat shrunk onto the mandrel and may, as a result, conform to the outer surface of the mandrel and define inner lumen 26 that has the cross-sectional shape of the mandrel.

After positioning inner liner 30 over the mandrel (90), structural support member 32 may be positioned over inner liner 30 (92). In examples in which structural support member 32 includes a coil member, the wire defining the coil member may be wound over an outer surface of inner liner 30 or pushed over inner liner 30. In some examples, the coil has any suitable shape (e.g., can be helical coil) before being positioned over inner liner 30, and conforms to the outer profile of inner liner 30 after it is positioned over inner liner 30. In other examples, the coil may have a shape that corresponds to the cross-sectional configuration of inner liner 30 and the mandrel.

In some examples, the structural configuration of structural support member 32 may be at least partially defined prior to being positioned over inner liner 30. For example, a shape memory wire (e.g., a nickel-titanium wire) or a wire of an otherwise heat-settable metal or alloy may be wound over a different mandrel (e.g., a “coil mandrel”) on which inner liner 30 is not present or over the mandrel (e.g., before inner liner 30 is positioned on the mandrel) to define at least one of the desired coil pitch, the desired coil diameter, the desired tapering profile (e.g., a continuous tapering or progressive tapering), or the desired length of structural support member 32, and then heat set to substantially hold its shape. The wire may then be subsequently unwound from the mandrel onto a reel or a bobbin, and then positioned over inner liner 30.

Structural support member 32 may be secured in place relative to inner liner 30 using any suitable technique. For example, member 32 may be adhered to inner liner 30, e.g., via support layer 36 that is applied over member 32 after member 32 is positioned over inner liner 30 or via support layer 36 that is applied over inner liner 30 prior to positioning structural support member 32 over inner liner 30. In addition to, or instead of, support layer 36, outer jacket 34 may be used to secure structural support member 32 to inner liner 30.

In the technique shown in FIG. 10, after structural support member 32 is positioned over inner liner (92), outer jacket 34 is positioned over an outer surface of structural support member (94). In some examples, outer jacket 34 is adhered to an outer surface of structural support member 32, e.g., an adhesive and/or a polymer may be applied to outer surface of member 20 prior to positioning outer jacket 34 over member 20 and then cured after outer jacket 34 is positioned over member 20. In addition to, or instead of, the adhesive, outer jacket 34 may be heat shrunk over member 20 and inner liner 30. In some examples, the heat shrinking of outer jacket 34 helps secure member 20 in place relative to inner liner 30.

In some examples, catheter 10 or catheter body 12 may be a part of an assembly that includes, e.g., a guidewire and/or another catheter. The catheter 10 or catheter body 12 in such an assembly can be any of the embodiments or examples of the catheter 10 or catheter body 12 disclosed herein. The guidewire may be used to guide catheter 10 to a target tissue site within the vasculature of a patient. In addition, in some examples, the additional catheter of the assembly may also be configured to guide catheter 10 or body 12 to a target tissue site within the vasculature of a patient. The additional catheter of the assembly may be substantially similar (e.g. identical or nearly identical) in construction to catheter 10 (including any of the embodiments or examples of the catheter 10 disclosed herein), but may have proportionally greater or smaller dimensions, such that the catheter bodies of the catheters may nest together. The assembly may therefore comprise the catheter 10 with the additional catheter positioned in the inner lumen 26 of the catheter, and may further comprise the guidewire positioned in the inner lumen of the additional catheter.

Each of the components of the assembly may be slidably disposed relative to the other(s) so that each may be advanced and/or retracted over or within the other(s). For example, when the additional catheter is positioned in the lumen of the catheter 10, the catheter 10 may be advanced or retracted longitudinally over the additional catheter, and/or the additional catheter can be advanced or retracted longitudinally within the catheter 10. The use of the additional catheter in this manner may help reduce any adverse interactions with tissue attributable to the ledge effect, as can occur when a relatively large catheter is advanced over a guidewire through a curve in a vessel and the distal rim of the catheter scrapes the vessel wall on the “outside” of the curve. For example, if in use of an assembly having a guidewire the guidewire is first advanced into the vasculature, the additional catheter may next be advanced over the guidewire before the catheter 10 is advanced over the additional catheter. The difference in outer diameter between the guidewire and the additional catheter (and between the additional catheter and the catheter 10) is less than the difference in outer diameter between the guidewire and the catheter 10. Therefore, any ledge effect arising from advancing the catheter 10 over a “bare” guidewire may be mitigated by use of the additional catheter in this manner. In other examples, the additional catheter of the assembly may have a larger outer diameter than catheter 10 or body 12 and may be guided over catheter 10 or body 12 to a target tissue site within the vasculature of the patient.

In some examples, a method of using catheter 10 (including, e.g., any of the embodiments or variants of the catheter disclosed herein) comprises introducing a guidewire or an inner catheter into vasculature (e.g., an intracranial blood vessel) of a patient via an access point (e.g., a femoral artery), and guiding catheter body 12 over the guidewire or the inner catheter. Once distal end 12B of catheter body 12 is positioned at the target tissue site, a medical procedure may be performed using catheter body 12. For example, another medical device (e.g., a stent, coil, thrombectomy device or endovascular retrieval device, or a delivery system used in combination with any of the foregoing) can be introduced through inner lumen 26 to reach the target tissue site. As another example, thromboembolic material may be aspirated from the vasculature by at least applying a vacuum force to inner lumen 26 of catheter body 12 via hub 14 (and/or proximal end 12A), which may cause the thromboembolic material to be introduced into inner lumen 26 via distal opening 13. Optionally, the vacuum or aspiration can be continued to thereby draw the thromboembolic material proximally along the inner lumen 26, all or part of the way to the proximal end 12A or hub 14. As a further option, the aspiration or vacuum may cause the thromboembolic material to attach or adhere to the distal tip of catheter body 12; in such a case the catheter 10 or catheter body 12 and the thromboembolic material can be withdrawn from the vasculature together as a unit, for example through another catheter that surrounds the catheter 10 or catheter body 12.

Various examples have been described. These and other examples are within the scope of the following claims.

What is claimed is:

1. A catheter comprising:
a catheter body extending between a proximal end and a distal end and defining at least one inner lumen, the catheter body comprising a proximal portion including the proximal end, a distal portion including the distal end, and a medial portion between the proximal and distal portions,
wherein a cross-section of at least one of the proximal portion or the medial potion, taken substantially orthogonal to a longitudinal axis of the catheter body, has a shape including at least three apexes, adjacent apexes being connected by curvilinear sidewalls.

2. The catheter of claim 1, wherein the at least three apexes is three apexes.

3. The catheter of claim 1, wherein the at least three apexes is four, five, six, seven or eight apexes.

4. The catheter of claim 1, wherein the shape is substantially symmetrical about the longitudinal axis of the catheter body.

5. The catheter of claim 1, wherein the curvilinear sidewalls are shaped substantially as arcs.

6. The catheter of claim 1, wherein each curvilinear sidewall is located between two of the apexes, and bows radially outwardly from the two apexes.

7. The catheter of claim 1, wherein an entire length of the catheter body has a cross-sectional shape including at least three apexes, adjacent apexes being connected by curvilinear sidewalls.

8. The catheter of claim 1, wherein a cross-sectional shape of the distal portion of the catheter body, taken substantially orthogonal to the longitudinal axis of the catheter body, has the shape including the at least three apexes, adjacent apexes being connected by curvilinear sidewalls

9. The catheter of claim 1, wherein a cross-section of the at least one lumen has the shape including at least three apexes, adjacent apexes being connected by curvilinear sidewalls.

10. The catheter of claim 1, wherein the catheter comprises a first catheter comprising a first catheter body, the first catheter body defining a protrusion extending into an inner lumen of the at least one inner lumen, the first catheter body being configured to receive second and third catheter bodies within the inner lumen on opposite sides of the protrusion.

11. The catheter of claim 1, wherein the catheter body comprises:
an inner liner;
a structural support member; and
an outer jacket, wherein the structural support member is positioned between the inner liner and the outer jacket.

12. The catheter of claim 1, wherein the proximal, distal, and medial portions have substantially equal lengths.

13. A system comprising:
a first catheter comprising a first catheter body extending between a proximal end and a distal end and defining a first inner lumen, the first catheter body comprising a proximal portion including the proximal end, a distal portion including the distal end, and a medial portion between the proximal and distal portions, wherein a cross-section of at least one of the proximal portion or the medial potion, taken substantially orthogonal to a longitudinal axis of the catheter body, has a shape including at least three apexes, adjacent apexes being connected by curvilinear sidewalls; and
a second catheter comprising a second catheter body defining a second inner lumen configured to receive the first catheter body.

14. The system of claim 13, wherein the proximal, distal, and medial portions of the first catheter are first proximal, medial, and distal portions, respectively, the proximal and distal ends of the first catheter body are first proximal and distal ends, respectively, and the second catheter body comprises a second proximal portion including a second proximal end of the second catheter body, a second distal portion including a second distal end of the second catheter body, and a second medial portion between the second proximal and distal portions, wherein a cross-section of at least one of the second proximal portion or the second medial portion, taken substantially orthogonal to a longitudinal axis of the second catheter body, has a substantially circular shape.

15. The system of claim 13, wherein the proximal, distal, and medial portions of the first catheter are first proximal, medial, and distal portions, respectively, the proximal and distal ends of the first catheter body are first proximal and distal ends, respectively, and the second catheter body comprises a second proximal portion including a second proximal end of the second catheter body, a second distal portion including a second distal end of the second catheter body, and a second medial portion between the second proximal and distal portions, wherein a cross-section of at least one of the second proximal portion or the second medial portion, taken substantially orthogonal to a longitudinal axis of the second catheter body, has the shape including at least three apexes, adjacent apexes being connected by curvilinear sidewalls.

16. The system of claim 13, wherein a cross-sectional shape of the distal portion of the first catheter body, taken substantially orthogonal to the longitudinal axis of the catheter body, has the shape including the at least three apexes, adjacent apexes being connected by curvilinear sidewalls.

17. The system of claim 13, wherein a cross-section of the first inner lumen has the shape including at least three apexes, adjacent apexes being connected by curvilinear sidewalls.

18. The system of claim 13, further comprising a third catheter comprising a third catheter body, wherein the second catheter body defines a protrusion extending into the second inner lumen, the second catheter body being configured to receive the first and third catheter bodies within the second inner lumen on opposite sides of the protrusion.

19. A method of forming a catheter, the method comprising:
positioning an inner liner over a mandrel, wherein a cross-section of at least one of a proximal portion of the mandrel or a medial portion of the mandrel, taken substantially orthogonal to a longitudinal axis of the mandrel, has a shape including at least three apexes, adjacent apexes being connected by curvilinear sidewalls, the mandrel comprising the proximal portion including a proximal end of the mandrel, a distal portion including a distal end of the mandrel, and the medial portion between the proximal and distal portions;
positioning a structural support member over the inner liner; and
positioning an outer jacket over the structural support member.

20. The method of claim 19, wherein an entire length of the mandrel has a cross-sectional shape including at least three apexes, adjacent apexes being connected by curvilinear sidewalls.

21. The method of claim 19, wherein a cross-section of the distal portion of the mandrel, taken substantially orthogonal to the longitudinal axis of the mandrel, has a substantially circular shape.

22. The method of claim 19, wherein a cross-sectional shape of the distal portion of the mandrel, taken substantially orthogonal to the longitudinal axis of the mandrel, has the shape including the at least three apexes, adjacent apexes being connected by curvilinear sidewalls.

23. The method of claim 19, wherein the mandrel defines an indentation configured to define a protrusion along at least a portion of the inner lumen.
